(19) United States
(12) Patent Application Publication
(10) Pub. No.: US 2021/0239545 A1
(43) Pub. Date: Aug. 5, 2021

(54) WIDE AREA SENSORS

(71) Applicant: Direct-C Limited, Edmonton (CA)

(72) Inventors: Adrian Banica, Edmonton (CA); Stephen Edmondson, Nobel (CA); Kaushik Parmar, Calgary (CA)

(21) Appl. No.: 17/049,299

(22) PCT Filed: Apr. 18, 2019
(86) PCT No.: PCT/CA19/50494

§ 371 Date: Oct. 20, 2020

Related U.S. Application Data
(60) Provisional application No. 62/660,931, filed on Apr. 20, 2018.

(51) Int. Cl. G01L 1/20; G01M 3/18

(52) U.S. Cl. CPC G01L 1/20; G01N 33/241; G01M 3/18

(57) ABSTRACT

A sensor has an at least partially conductive material to which plural conductive contacts are connected. A processor causes circuits to be formed between pairs of the conductive contacts to measure an electrical property of the material between the contacts, such as a resistance of the material. The processor conducts such measurements between plural pairs of contacts to determine a location of an influence on the material and produces an output indicating the location. The influence can be, for example, a disruption of the material or, where the electrical properties of the material are affected by strain, a strain of the material.

### PRIORITY CLAIM

This application is a U.S. national phase of International Patent Application No. PCT/CA19/50494 filed Apr. 18, 2019; which claims priority from U.S. Provisional Patent Application No. 62/660,931 filed Apr. 20, 2018, the contents of which are incorporated herein by reference in their entireties.

### FIELD OF THE INVENTION

Sensor Coatings.

### BACKGROUND OF THE INVENTION

In international patent application PCT/CA2018/050013 a sensor coating is described. The sensor coating uses a material with resistance sensitive to influences, and probed using a mesh in the sensor coating to detect and locate variations in the resistance.

An example coating composition is disclosed PCT/CA2014/050992.

The polymer based smart coating contains nanoparticles and is sensitive to the presence of hydrocarbons. It is used to monitor and detect leaks of hydrocarbons from storage units such as tanks and pipes by monitoring changes in resistance. Monitoring of stress and strain and cracks is also possible with these smart coatings. Wireless communication can be used with the sensor for remote monitoring.

It would however be desirable not to require mesh at the detection area of a sensor coating.

### SUMMARY OF THE INVENTION

There is provided a sensor for detecting an influence of a detection area of a sheet or volume of an at least partially conductive material. The sensor can comprise plural conductive contacts arranged in electrical contact with the sheet or volume of the at least partially conductive material around the detection area, and a processor connected to the conductive contacts. The processor can be configured to form or energize electrical circuits connecting pairs of the conductive contacts through the sheet or volume of at least partially conductive material. It can measure an electrical property of these electrical circuits. The processor can detect a location of the influence, and produce an output accordingly, based on the measurements of the electrical property for the pairs of conductive contacts.

These and other aspects of the device and method are set out in the claims.

### BRIEF DESCRIPTION OF THE DRAWINGS

Preferred and alternative examples of the present invention are described in detail below with reference to the following drawings.

Embodiments will now be described with reference to the figures, in which like reference characters denote like elements, by way of example, and in which:

FIG. 1 is a top view of an exemplary sensor board with large contacts.

FIG. 2 is a top view of an exemplary sensor board with small contacts.

FIG. 3 is a picture showing a board with small contacts and a board with large contacts, each having a disruption of the sensor material, and showing a path between contacts on perpendicular sides of the board with small contacts.

FIG. 4 is a picture showing a sensor board having a disruption, and showing paths between contacts on parallel sides of the board.

FIG. 5 is a schematic diagram of an exemplary sensor for detecting pill removals from a blister pack.

FIG. 6 is a schematic diagram showing an expected contour map expected to be produced using the sensor of FIG. 5.

FIG. 7 is a schematic diagram of an exemplary sensor in which each contact point is controlled by a node.

FIG. 8 is a schematic diagram showing an exemplary version of a node suitable for the sensor of FIG. 7.

FIG. 9 is a schematic diagram showing an exemplary version of a node suitable for a wireless version of the sensor of FIG. 7.

FIG. 10 is a graph showing reduction of power production of a wind turbine due to erosion.

FIG. 11 is a side cross section schematic view of a coating for a wind turbine blade showing layers of the wind turbine and embedded sensor.

FIG. 12 is a top view of a sensor for a wind turbine blade.

FIG. 13 is an end cross section schematic view of a wind turbine blade showing sensor components separated from the blade for clarity, and omitting other layers of the blade.

FIG. 14 is a top view of a simpler sensor design for a hydrocarbon pipeline.

FIG. 15 is a top view of a 3D sensor showing the sensor material as transparent.

FIG. 16 is an end cross section view of the 3D sensor of FIG. 15.

### DETAILED DESCRIPTION OF THE PREFERRED EMBODIMENT

Immaterial modifications may be made to the embodiments described here without departing from what is covered by the claims.

A sensor is provided that measures stimuli using variations of an electrical property, such as resistance variations. The property is that of a sheet or volume of partially conductive material, which may, for example, be at a surface where influences are to be measured. Conductive contacts are arranged around a detection area of the surface to detect an influence on the detection area. A processor is connected to the conductive contacts and configured to measure resistance between pairs of the conductive contacts, and produce an output indicating a location of the influence based on the pairs of resistances. The influence can be a disruption in the at least partially conductive surface. It can also be something that modifies the conductivity of the surface. The surface may be selected to have a resistance that is altered by various influences, such as strain, temperature and the presence of a fluid. A surface with electrical properties modified by influences to be detected is referred to as a “smart coating”.

A smart coating can be of any suitable thicknesses including a thin film (for example 30 micron). The sensor can be of any size and have any number of sensing zones through measurements between different contacts.

Testing for SmartBlister concept on Rigid Sensor Boards

Several boards were coated with a semi-conductive polymer nano-composite coating and the change in resistance was measured as the coating was removed to form a discontinuity in the smart coating. The shape of the discontinuity in these tests corresponded to a shape expected in the event of the removal of a pill from a blister pack, in a shape corresponding to the removal of a pill. The coating removal thus simulates a pill removal event when the film is broken leading to a loss of continuity in the coating film, suitable for a blister pack embodiment as described below. However, such disruptions of the partially conductive surface may also result from other causes, such as wear of a surface in which the partially conductive surface is embedded, or cracking of an underlying structure to which the partially conductive surface is attached.

The tests used 120 mm×70 mm rigid PCB board with 30 contacts spaced around the circumference every 10 mm effectively building a 100×50 mm detection area. The coating used included an existing nanocomposite coating formulation referred to as ST04 (containing carbon nanotubes and graphene nanoplatelets) and used in a hydrocarbon sensor system such as the system described in PCT/CA2018/050013. ST04 coating is optimized for Hydrocarbon sensing but it is piezoresistive so it will respond to physical stimuli and can detect stress, strain and cracks. It has a relatively high resistance when applied across such a large area.

Another coating referred to as ST05 was used in other tests. The ST05 is also hydrocarbon sensitive.

In these tests described in this document an Agilent 10252A digital multi-meter (DMM), capable of measuring resistances up to 500 Mohms, was used for reading the coating resistance values between contacts.

In this document “contacts” refers to the points between which electrical properties are measured. This measurement can comprise touching leads to the contacts, or by, e.g., mechanical or solid state switching without the movement of any leads. The terms “sensor points” or “measurement points” are used interchangeably with “contacts”.

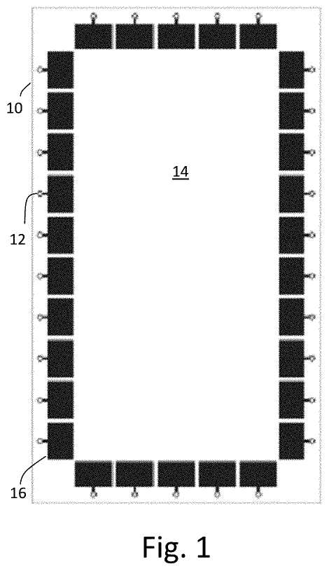

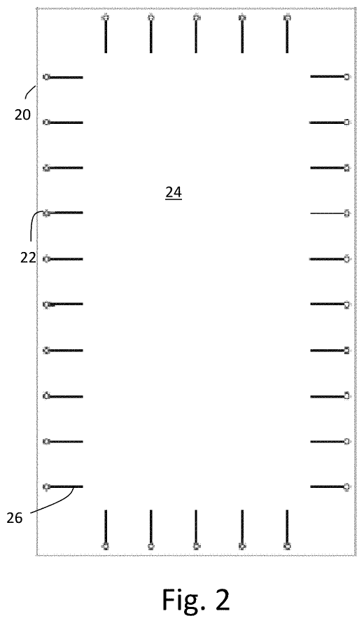

The boards either had large or small contacts arranged around the detection area. FIG. 1 shows a board 10 coated with a smart coating (not shown in FIGS. 1 and 2) and having large with large contacts 12 arranged around and defining a detection area 14. The large contacts comprise pads 16 that are attached to a rigid surface of the board, and the coating is sprayed over the pads to cover both the detection area 14 and pads 16. Thus, the pads would be under the smart coating when the coating is applied.

FIG. 2 shows a board 20 having small contacts 22 arranged around a detection area 24. The small contacts include ends 26 that would be under the smart coating in this embodiment when applied. For either of these embodiments, the contacts could also be placed over the coating if desired.

In FIGS. 1-2 the contacts are arranged rectilinearly and define rows and columns. The contacts need not define rows and columns and resistance can be measured between any pair of contacts. Indeed, each contact can be part of multiple pairs between which the resistance is measured to map the resistance in more detail.

The contacts are arranged in this embodiment in 5 columns numbered in order from 1 to 5 and 10 rows numbered in order from 1 to 10. Column contacts at the top are referred to here as CA1-CA5 depending on the column number and at the bottom are referred to as CB1-CB5 depending on the column number. Likewise row contacts at the left are referred to as RA1-RA10 depending on the row number and row contacts at the right are referred to as RB1-RB10 depending on the row number. Table 1 shows a map of these locations.

TABLE 1
Sensor Location Map

| | CA1 | CA2 | CA3 | CA4 | CA5 | |
|---|---|---|---|---|---|---|
| RA1 | | | | | | RB1 |
| RA2 | | | | | | RB2 |
| RA3 | | | | | | RB3 |
| RA4 | | | | | | RB4 |
| RA5 | | | | | | RB5 |
| RA6 | | | | | | RB6 |
| RA7 | | | | | | RB7 |
| RA8 | | | | | | RB8 |
| RA9 | | | | | | RB9 |
| RA10 | | | | | | RB10 |
| | CB1 | CB2 | CB3 | CB4 | CB5 | |

A preliminary scratch test using a sensor with the ST04 coating was performed where two directly opposite contacts were connected to the DMM and the resistance recorded along the 50 mm coating path. The coating was scratched and removed along a direct line between the two measurement points in three different locations. This process was repeated twice. In a first test, the coating was scratched near row 2. The initial resistance between the row 2 contact points was 140 MOhms. A scratch was added in the middle resulting in 180 Mohms. Successive scratches on each side of the middle scratch on the direct line resulted in 250 and 379 Mohms.

Resistance was then measured between the row 5 contact points and scratches added in the direct line between these points. The initial resistance was 200 MOhms. A scratch towards one end of the direct line resulted in 280 MOhms. A scratch towards the other end brought it to 340 Mohms, and a middle scratch resulted in 385 MOhms.

In a further test, resistance was measured between contact points at row 7 of the same board. The initial resistance was 445 MOhms. This time some pressure was applied using one finger to the coating and then removed, then a scratch was made in the coating. As the pressure was applied, 422 MOhms was measured. The pressure was increased, resulting in 413 MOhms. The pressure was then removed resulting in 473 MOhms. A scratch was added in the middle of the direct line of the contacts resulting in 507 MOhms. A further scratch added in the direct line increased the resistance over the range of the particular resistance measurement tool used.

Further boards were made using the ST05 coating. In a set of tests the change along the rows and columns was measured between parallel sensor points. That is, the resistance was measured between pairs of contacts, each pair defining a row or column.

In a first test, with data shown in Table 2, scratches were formed in row 1 at different columns. Resistance was measured between pairs of sensors defining a column, for example CA1 and CB1 to measure the resistance for column 1. Each row in the table provides data for a different (or no) scratch. Resistance changes are measured relative to the baseline with no scratches.

TABLE 2
Change in Resistance for Columns
as Scratches added Large Sensors

| | Scratch | | | | | |
|---|---|---|---|---|---|---|
| | Location | Col. 1 | Col. 2 | Col. 3 | Col. 4 | Col. 5 |
| | none | 0.0 | 0.0 | 0.0 | 0.0 | 0.0 |
| Test 1 | R1/C1 | −0.4 | 0.0 | 0.0 | 0.0 | 0.0 |
| Test 2 | R1/C2 | 0.8 | 0.9 | 0.4 | 0.4 | 0.3 |
| Test 3 | R1/C3 | 0.4 | 0.5 | 0.6 | 0.4 | 0.3 |
| Test 4 | R1/C4 | 0.1 | 0.3 | 0.7 | 0.9 | 0.5 |

The location of the coating loss can be detected, it corresponds to the column with the largest change in resistance (in bold).

In a second test, with data shown in Table 3, scratches were formed in row 1 at different columns. Resistance was measured between pairs of sensors defining a row, for example RA1 and RB1 to measure the resistance for row 1. The table shows the change in resistance as scratches were added.

TABLE 3

| Scratch location | R1 | R2 | R3 | R4 | R5 | R6 | R7 | R8 | R9 | R10 |
|---|---|---|---|---|---|---|---|---|---|---|
| None | 0.0 | 0.0 | 0.0 | 0.0 | 0.0 | 0.0 | 0.0 | 0.0 | 0.0 | 0.0 |
| R1/C1 | −0.2 | 0.0 | 0.0 | 0.0 | 0.0 | 0.0 | 0.0 | 0.0 | 0.0 | 0.0 |
| R1/C2 | 0.8 | 0.6 | 0.5 | 0.3 | 0.3 | 0.2 | 0.2 | 0.1 | 0.1 | 0.2 |
| R1/C3 | 1.3 | 0.9 | 0.8 | 0.5 | 0.4 | 0.3 | 0.3 | 0.2 | 0.2 | 0.3 |
| R1/C4 | 1.4 | 1.0 | 0.9 | 0.6 | 0.5 | 0.4 | 0.4 | 0.4 | 0.3 | 0.4 |

The location of the coating loss can be detected by measuring resistance change along the Rows, it corresponds to the Row with the largest change in resistance, Row 1 in each case. Consistent resistance across the entire board is preferred but this data shows that this method can work even where there is variation in the resistance across the board surface.

Contour Plot Mapping

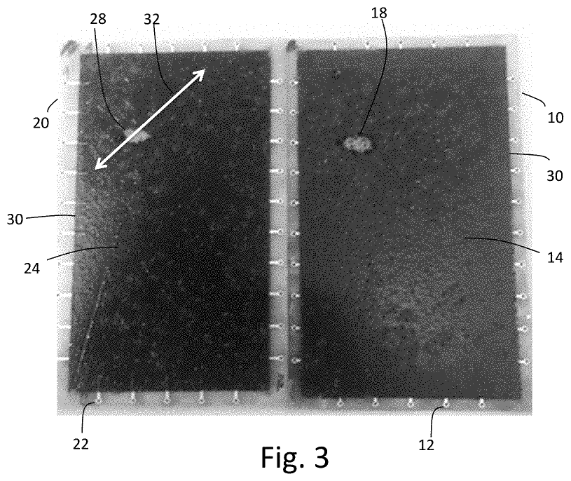

In further tests the resistance was mapped either between perpendicular sensor locations (sensors configured at a 90 degree angle to each other, measured from columns to rows such as RA to CA) or between parallel sensors (CA to CB), which can be from the same row or not. The resistance was then mapped again after one scratch was added. This was done for both large and small sensors. FIG. 3 shows a board 20 with small sensors and a board 10 with large sensors side by side. Smart coating 30 is shown on the boards covering the detection areas 14 and 24, and scratches 18 in the large sensor board 10 and 28 in the small sensor board 20 are shown.

Table 4 shows data from a test measuring the resistance change for each pair of contacts comprising a left hand side row contact and a top column contact, when one scratch is added. This test uses the board 10 with large contacts. The percentage resistance change is shown.

TABLE 4

| | CA1 | CA2 | CA3 | CA4 | CA5 |
|---|---|---|---|---|---|
| RA1 | 3 | 2 | 2 | 1 | 1 |
| RA2 | 2 | 2 | 2 | 1 | 1 |
| RA3 | 2 | 2 | 2 | 1 | 1 |
| RA4 | 2 | 2 | 2 | 1 | 1 |
| RA5 | 2 | 1 | 1 | 1 | 1 |
| RA6 | 1 | 1 | 1 | 1 | 1 |
| RA7 | 1 | 1 | 1 | 1 | 1 |
| RA8 | 1 | 1 | 1 | 1 | 1 |
| RA9 | 1 | 1 | 1 | 1 | 1 |
| RA10 | 1 | 1 | 1 | 1 | 1 |

Table 5 shows a corresponding test for the board 20 with small contacts. This table shows the percent change of resistance between the perpendicular sensor locations tested, when one scratch is added.

TABLE 5

| | CA1 | CA2 | CA3 | CA4 | CA5 |
|---|---|---|---|---|---|
| RA1 | 8 | 5 | 8 | 7 | 6 |
| RA2 | 3 | 2 | 4 | 2 | 2 |
| RA3 | 5 | 6 | 8 | 9 | 6 |
| RA4 | 16 | 20 | 22 | 23 | 18 |
| RA5 | 4 | 3 | 5 | 3 | 2 |
| RA6 | 4 | 3 | 5 | 4 | 3 |
| RA7 | 3 | 3 | 4 | 2 | 1 |
| RA8 | 3 | 2 | 3 | 2 | 2 |
| RA9 | 3 | 3 | 4 | 3 | 2 |
| RA10 | 4 | 5 | 5 | 5 | 4 |

Based on Tables 4 and 5, the small sensors can be seen to give seen to give a much larger change in resistance when some coating is removed. It is believed that this is due to the larger area of at least partially conductive coating between the sensors which results in a larger variation in resistance. It is likely there is an optimum size, too small and results will be inconsistent due to the small contact area between the sensor and the coating, too large and the variation obtained when a discontinuity is generated will be too small.

Of the paths between contact pairs, each contact pair comprising a left row contact and a top column contact (the change of resistance of which is shown in Table 5), the shortest connected path that intersects with the defect is CA4 to RA4, as shown by arrow 32 in FIG. 3, so this gives the highest change in resistance. Thus, the location of the defect can be inferred as being on this shortest path—this analysis is based on the individual values of change in resistance, no interpolation is required.

A contour plot (not shown) was generated based on Table 5. On this contour plot, both the location and shape of the defect could be seen. Based on examining the points of highest resistance, it can be seen that it is located along the path of highest resistance, thus between RA4 and the Row of Sensors on CA1-CA5. The coating defect is seen to start close to a line that connects RA4 and CA2 and ends at a line that connects RA4 and CA5. Thus, using readings from sensors perpendicular to each other, location information can be determined, as can the blister shape. A simple Excel contour graph plot thus shows the location and shape of the blister based on single resistance readings taken from perpendicular sensors only and mapped to form a contour plot. This uses interpolation (and can use smoothing) to get a better visualization of the location of resistance increase. This data can be displayed in other ways such as using a heat map or 3D visualization.

In a further test, comparisons were taken between parallel sensors, in this case different pairs of one top and one bottom column sensor each. The table shows the percentage resistance change when one scratch is added. This test used a board with small contacts.

TABLE 6

| | CA1 | CA2 | CA3 | CA4 | CA5 |
|---|---|---|---|---|---|
| CB1 | 3.30 | 3.15 | 2.88 | 3.00 | 1.75 |
| CB2 | 3.09 | 3.28 | 3.00 | 3.16 | 1.83 |
| CB3 | 2.96 | 2.70 | 2.43 | 2.45 | 1.72 |
| CB4 | 2.88 | 2.61 | 2.34 | 2.35 | 1.66 |
| CB5 | 2.48 | 2.47 | 2.21 | 2.19 | 1.17 |

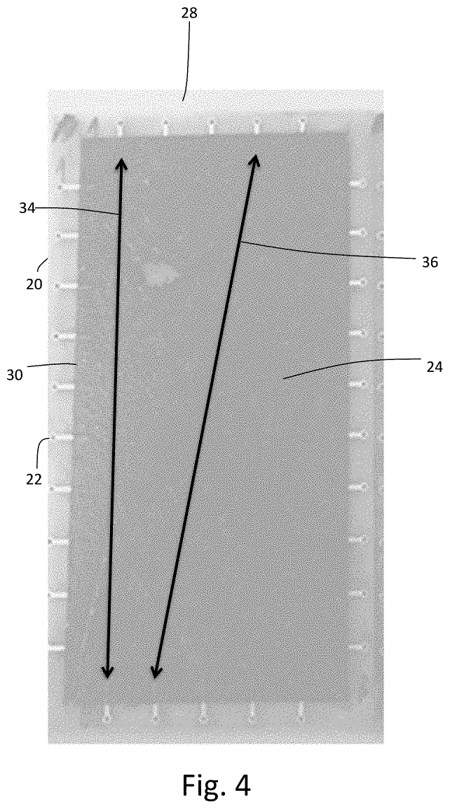

Examining readings taken from parallel sensors (pairs of contacts on opposite sides of the detection area) in Table 6, the location of the defect can also be inferred, however the location cannot be determined with as much accuracy as it can using perpendicular sensors. See FIG. 4, the location of scratch 28 is between the arrows 34 and 36, but the exact location cannot be fixed, since there is not enough discrimination between the change in resistance values. Thus, using non-parallel sensors such as perpendicular sensors is preferred to give more accurate detection of the location of any smart coating defects.

Parallel sensors can still be used. There is a tradeoff between number of measurements used amount of detail obtained. Simply measuring across the board to sensors that are parallel to each sensor gives information on crack location with only a few measurements.

Conclusions from Testing Described Above
The removal of polymer nano-composite coating along a straight-line path on a 2D surface can be detected by monitoring the resistance along that path with a simple multi-meter using low voltage DC power.
Using multi-point analysis, measuring resistance from point to point for all of the points, a more detailed contour map could be drawn up.
Using small sensor bars gives a greater percentage change in resistance as coating is removed vs. large sensor bars.
Multiple removals of coating along that same line can be detected, with the resistance progressively getting higher as more coating is removed.
The area where there is a discontinuity in the coating can be inferred from the position of maximum change in resistance on contour map corresponding to an intersection point for perpendicular sensors.
A mild stress such as the pressure generated by one finger can be detected by the coating on a flexible substrate by monitoring the change in resistance.

Blister Pack Pill Removal Detection

In some embodiments, the sensor is used to detect a removal of pills from a package.

It is expected that smart blister-based packaging will be rolled out by Pharmaceutical companies for several high cost drugs over the next 5 years. For example a smart package for a cancer drug is planned using a custom wired solution where there is one specially formed wire for each blister.

If instead of wires, a smart coating was used to cover the entire area where the blisters are present, the smart coating offers the advantage of being able to sense more than a wired system, since, with an appropriate choice of material, it can detect temperature and stress/strain. It can also be much simpler than the custom wiring that they now employ. The same smart coating configuration, with a consistent arrangement of contacts around a detection area, could be used for different blister pack configurations as long as the blister pack configurations fit within the detection area.

A thin film smart coating should result in a cost close to the wired solution.

The smart coating would detect a pill opening event and could be mapped to show where pills have previously been removed.

Some pills are in a folding blister pack with pills in both halves of the pack, here the smart coating could be used as an adhesive layer that connects the two halves of the pack.

A smart coating is applied at the back of a pharmaceutical blister pack to provide a detection area. It could also be applied to the front. Pills are removed from the blister pack by pushing them through the back of the blister pack in pre-defined locations within the detection area. The smart coating is first disturbed by the pressure applied to move the pill and then breaks as the pill travels through the coating. Thus, the removal of the pill would be detected by monitoring the coating as the pill is removed and a map could be drawn showing where pills have previously been removed by interrogating the Smart Coating. The pill locations can be detected using an electronic signature as the individual blister pack locations are opened, without the need to break a circuit for each location.

A Smart Coating which contains nanoparticles so that it gives a strong, piezoresistive response to the force exerted is useful to detect pill removal. The CA2014050992 patent composition is a suitable example. A resistivity that gives a semi-conductive film is also preferred for monitoring of the coating after removal. 20-200 kOhm across the sensors is preferred across the gap between the sensors.

The back of the blister pack may also comprise foil which the smart coating is electrically insulated from. The sensor can comprise a processor which can communicate with an electronic device such as a smart phone using Near Field communication.

The processor can detect changes in the properties of a large area of coating using contacts arranged around the detection area. This simplifies the wiring required compared to sensors that requires wires to be broken. The same coating/sensor configuration can be employed for different blister pack configurations, it will simply create a map showing wherever a pill has been removed, based on changes since the map was last drawn, so sequential removal of pills can be detected. It does not need to be configured to a particular blister pack configuration.

The smart coating may be an adhesive layer that bonds two layers of the pill package together. The package may be a bi-fold package with two sets of blister packs that are connected by a smart coating layer.

Crucially, all of the sensing wires and any required connecting wires can be located outside of the sensing area, the sensing area need only comprise Smart Coating and, if needed, backing materials.

For pouch based packaging, where medications are contained in a series of pouches in a strip configuration, a smart coating can be applied to the back of each pouch, either as a continuous film or as a series of bands. The resistance of these bands is monitored, and the number of opened pouches can be determined based on the increase in resistance.

Pill extraction can detected based on a sensed event, for example the removal of the pill can be inferred based on an electronic signature of the coating being disturbed as the individual blister pack locations are opened.

This can be done in several ways, for example, in one embodiment a Gravity/tilt sensor detects movement of the package and wakes up the monitoring circuit. In another embodiment the removal can be determined solely by measurements of resistance. In an example, a circuit measures the smart coating by interrogating across sensor points, and detects changes in resistance due to one or more of temperature change as package is handled, or a change in the strain/tear of the coating as the pill is pushed, first of all stretching and then breaking through the film. An algorithm based on change in temperature and strain of coating can be used to confirm that pill has been pushed out, and a processor at the package or at a receiver such as a cellphone can record the time that the pill is removed.

Once a pill extraction event has been identified, the circuit can send a signal indicating which pill locations have now been opened.

The circuit interrogates the smart coating and builds a map showing the change in resistance, indicating which pill locations have now been opened. As coating is removed, the resistivity changes. A consistent resistivity across the board is preferred to give accurate mapping. Variation of resistivity will give less accurate results.

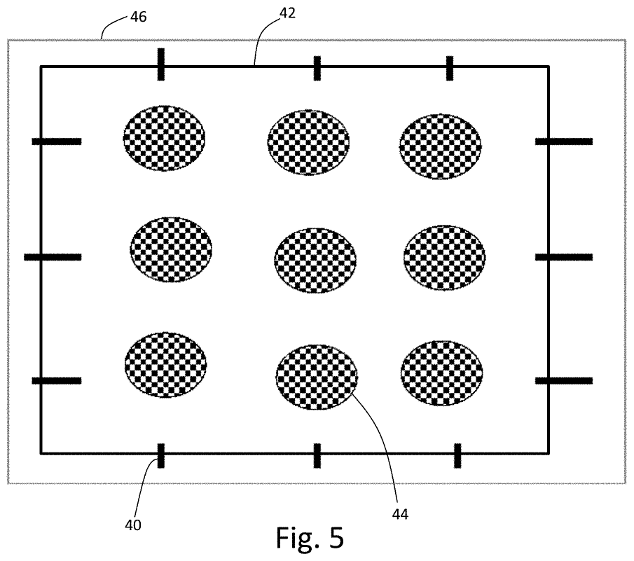

In a simple example shown in FIG. 5, a 3×3 blister pack is used with resistance measured along 3 rows and 3 columns using sensor points 40 at the end of each row or column. A smart coating 42 overlaps at least part of each sensor point 40 and covers the blisters 44 which are in the detection area between the sensor points 40. The smart coating is continuous in this embodiment, but it could also include discontinuities as the system can still detect changes as discontinuities are added or widened. A non-conductive backing 46 can support the smart coating and the contacts 40.

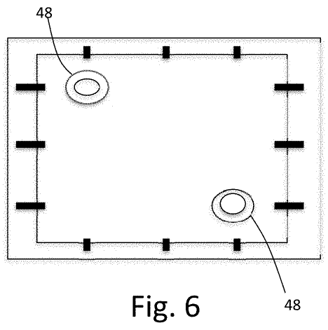

An example predicted output map after two pills are extracted is shown in FIG. 6. In this example, a contour map shows two increased resistance locations 48 indicating where pills were extracted. The contour map is overlaid on a representation of the sensor for better visualization of what the locations correspond to.

Large Area Sensing and Switchable Sensors

When conducting a measurement, a processor may enable two measurement points at a time and read the resistance between the two and then continue on until it completes all possible measurement pairs. This is simple when we have a small array (say 5×5) over a relatively small physical area but this becomes very problematic when we deal with very large areas (such as the side of a building) and lots of measurement points. This is because under the simplistic measurement method, each contact point would have to be connected to the controller input by a separate wire and if we have 100's of nodes over a large area, the wiring will be prohibitive.

Therefore, nodes are provided, each node being an element comprising switching circuitry connected to one or more of the conductive contacts. The processor is connected to send signals to one or more nodes to cause the one or more nodes to close a circuit through a pair of conducting contacts and the partially conductive material on the detecting area to measure a resistance between the pair of conductive contacts.

The concept behind the switching nodes is that each node will have a small circuit that can be commanded to turn its measurement location on or off and effectively connect it to one of two common measurement wires. One of the nodes would be commanded to connect its contact point to one wire and another one to the second wire after which the controller will measure the resistance between the two. The process will be repeated until the desired measurement nodes are completed.

As described each node controls signals a single contact point. Each node could also control multiple contact points. Where one node controls multiple contact points, the processor could be configured to only measure resistance between contact points connected to different nodes, or it could also measure resistance between contact points connected to a common node.

The advantage to this method compared to just using wires is that any number of measurements nodes can be accommodated with a bundle of, for example, 5-6 wires (2 for the measurement, 3-4 for power and a serial communication bus) compared to tens or hundreds of wires in the traditional way. The number of wires could be reduced, for example, by the nodes drawing power from the measurement or communication wires.

This system can also be used with a Near Field Communication (or some other radio protocol) configuration. In one such embodiment, this communication is used to eliminate the communication wires. In a further embodiment, each node can be interrogated using a radio signal with no wiring required at all. For example, a first signal can be sent to a node, to cause it to produce an AC signal between a contact point of the node and another contact point of the system. This AC signal can propagate between nodes connected by the at least partially conductive material even if the circuit connecting the nodes through the contacts is open. This can be, for example a resonant AC signal supported by inductive elements connected to the contacts by their respective nodes in response to the first signal. The frequency of resonance of the AC signal can be measured along with current and voltage. From this, electrical properties of the coating between the contact points can be measured. The electrical properties can include resistance and capacitance. The node can send a second signal back to the processor based on the results. In an example, the second signal is the resonant AC signal itself or a transmission using this signal. The properties can be determined using this second signal. In an embodiment, the AC signal can be directly produced by the received first signal, and the resonance is measured by varying the first signal and measuring the response of the second signal.

Thus a change in the resistance or capacitance of the coating can alter a resonant AC signal between two contact points which is directly or indirectly received at the processor to detect the change.

In other embodiments, the node includes a processor of its own to send the second signal based on the results of the measurement. The AC signal can also be non-resonant.

In any of these embodiments, the nodes can be powered by the transmitted first signals.

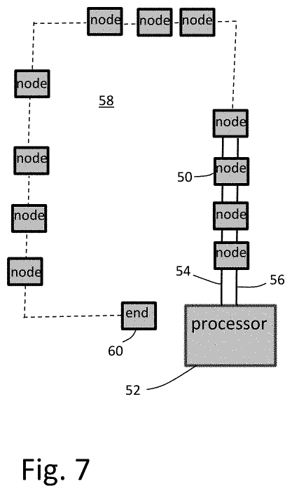

FIG. 7 is a sketch of a wired version of this concept showing a processor controlling many nodes 50, each node in this example having one sensor point (not shown). The nodes are connected to a processor 52 in this example using measurement wires 54, which can be for example two wires, of which only a single wire is shown, and addressing wires 56, which can for example be three wires, of which only a single wire is shown. The wires are only shown for the first few nodes; additional nodes are linked using dotted lines to indicate that the wires continue through these nodes and that many more nodes may be included and are not shown. The nodes surround a detection area 58.

In the embodiment shown there is an end node 60. In another embodiment, the nodes could be connected back to the processor in a loop. This could be used to eliminate one of the measurement wires if the nodes can cut off the measurement wire between the nodes. In a further embodiment, all measurements could be between a contact point controlled by a node of one set of nodes and a contact point controlled by a node of another set, each set connected to a separate single measurement wire.

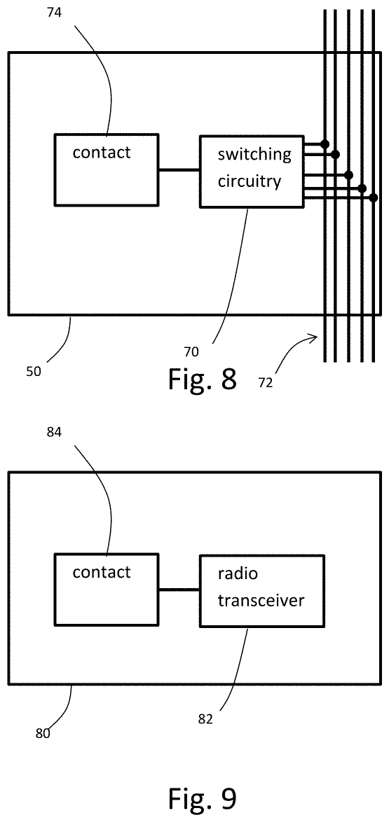

FIG. 8 is a schematic diagram showing an exemplary node 50. Switching circuitry 70 connects wires 72 to sensor point 74.

FIG. 9 is a schematic diagram showing an exemplary node 80 for a radio embodiment. A radio transceiver 82 receives a first signal to produce an AC signal between contact point 84 and another contact point, and sends a second signal based on the AC signal.

While these arrangements are contemplated for the case of areas with large numbers of sensing points, typically large sensing areas, they could also be used for areas with small numbers of sensing points and small sensing areas.

Examples of possible large sensing areas include infrastructure surface areas, such as the side of a building or a bridge.

Large Area Sensor for Cracking of Substrate or Loss of Coating

Using a distributed sensor array as described here and a semi-conductive smart coating, the surface of an infrastructure component such as a bridge or a wind turbine blade can be monitored. The coating will detect stress and strain caused by applied pressure such as the start of crack formation in, or deformation of, the substrate. It can also detect changes in size of existing cracks. The sensor can detect smart coating discontinuities such as cracks in the coating caused by cracking of the substrate or defects due to the loss of smart coating whether caused by environmental erosion or by some other mechanism.

The distributed sensor array can be turned on only when a reading is required, or run continuously. In both cases, the data can be sent via a radio transmitter to a modem and thence to a cloud based data collection and analysis software package. The contact points may be parts of nodes connected by wires or wirelessly. Where the nodes are connected by wires, they can still be controlled by a processor which is connected wirelessly to other equipment such as a cell phone.

Sensors can be deployed in either a 2D array measuring X and Y changes in the coatings or in a 3D array measuring X, Y and Z changes in the smart coating. Both approaches use distributed sensors to build a map of the coating and to monitor for changes in the coating based on the change in resistance caused by stimuli to be detected. The stimuli can include, for example, pressure, temperature, stress/strain or loss of continuity in the smart coating.

Wind Turbine Blades

For wind turbines, the main problem in service is leading edge erosion, caused by rain and dust impacting the leading edge of the blade. This is always worst at the end with the highest rotational speed, away from the hub, thus it only needs to be monitored over a relatively small area, close to that end. A wind turbine blade (WTB) coating system employed contains three layers, on top of the Glass Reinforced Epoxy (GRE) blade: epoxy primer, topcoat and Leading Edge Protection (LEP) layer. Currently the top coat and LEP are polyurethane coatings.

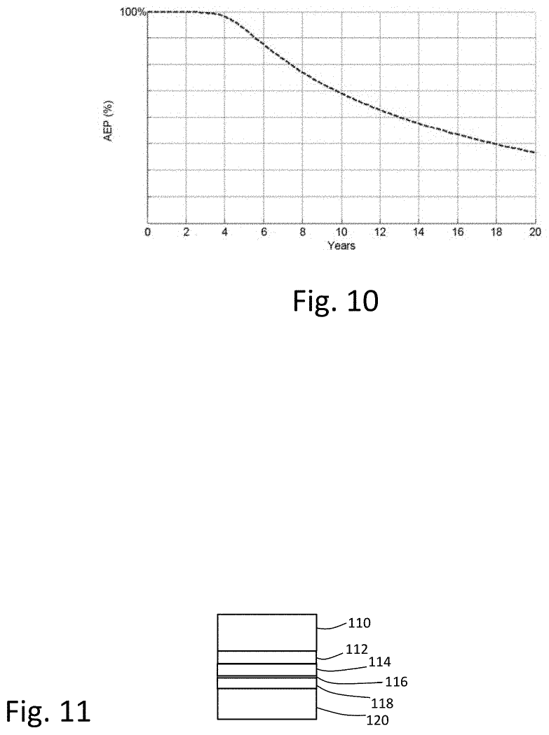

FIG. 10 is a graph showing the loss of performance in Annual Energy Production due to erosion of the LEP. It becomes a significant problem after only 7 years of the 20 year life span. Eventually, the blade can crack once the LEP and coating is eroded completely and the GRE itself starts eroding.

As the industry looks at better ways of protecting the leading edge from erosion, some monitoring of that leading edge would clearly be of benefit. A study used LIDAR to measure performance uplift after repair to moderately eroded blades and saw a 1.5 to 2% uplift in AEP.

Smart Coating Based Sensor for Monitoring Leading Edge (LE) Erosion

A sensor system may comprise a thin film sensor strip with contacts and connecting wires printed on with a smart coating applied on top of it to complete the sensing circuit.

It would be placed on top of the epoxy primer, so the smart coating is covered by the top coat and the LEP layer. FIG. 11 is a schematic diagram showing these layers.

In FIG. 11, LEP layer 110 is above top coat 112, which is on smart coating 114. Below this is sensor strip 116, which in turn is on epoxy primer 118, on GRE 120.

The smart coating would be exposed as soon as both the LEP and top coat layers were eroded, and would measure the erosion or cracking of the smart coating creating a 2D map of the eroded area of smart coating.

The sensor would be configured such that the sensing area would be exposed to erosion but none of the wiring would.

The sensor can be configured such that the detection area of the smart coating that is sensing these changes is in the area of a wind turbine blade where erosion is expected to occur. All of the conductive filaments, both sensing wires and connecting wires can be located away from this detection area, to ensure that they are not eroded or subject to cracking.

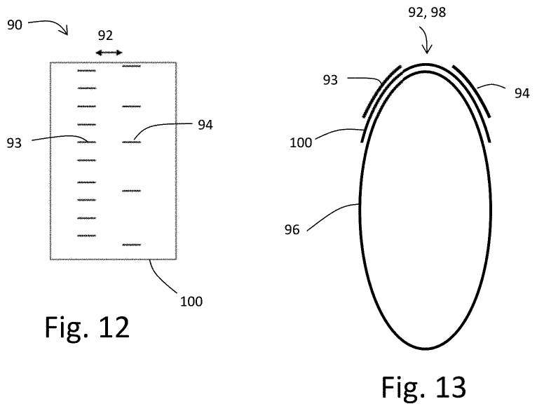

FIG. 12 is a schematic diagram showing a top view of sensor 90 when laid flat prior to installation showing sensing conductive filament points 93 and 94 at either side of a leading edge of a wind turbine blade. The sensing (detection) area 92 is at the leading edge with contacts 93 and 94 at either side of the leading edge. FIG. 13 is a schematic diagram showing this sensor on wind turbine blade 96 showing position of leading edge 98. The coating 100 and contacts 94 are shown separated from the blade 96 for clarity but would be attached as part of a blade coating.

Sensing filaments only are shown, connecting wires are not shown. Sensing elements could be switchable electronic circuits which are only switched on when a measurement is required. The wiring including contact points and, depending on the embodiment, switching elements, may be applied to the blade prior to the spraying of the smart coating. In an embodiment the sensor may communicate to a control box housed inside the turbine housing using Near Field communication.

The sensor array can indicate, via analysis in software, for example via a cloud based software package:

1. When the smart coating layer is exposed by loss of outer, protective coating layers. This would be by detection of pressure stimulus or the presence of a conductive solution such as salt water. A Smart Coating that gives a strong piezoresistive response to the stimulus is required. An example is a coating which contains nanoparticles such as the '992 patent composition.

2. The volume of smart coating that has been lost. It must have the correct resistivity such that it gives a semi-conductive film (20-200 kOhm measurement across the sensors is preferred), for monitoring of the volume of coating removed.

3—Where in the sensing area there is loss of continuity in the smart coating occurs either due to cracks or to coating loss due to erosion. This can be shown on a 2D or 3D map of the smart coating sensing area. A coating with consistent resistivity, lacking any areas with significantly higher or lower resistivity is preferred to give an accurate map.

The sensor can also be used in other contexts such as on oil pipe welds. For oil pipe welds, a swellable polymer can be incorporated into the sensor to detect Hydrocarbon leakage in addition to the other changes (such as the coating composition from the '992 patent).

A sensor for a pipe weld may be as shown in FIGS. 12 and 13 with the pipe weld taking the place of the leading edge.

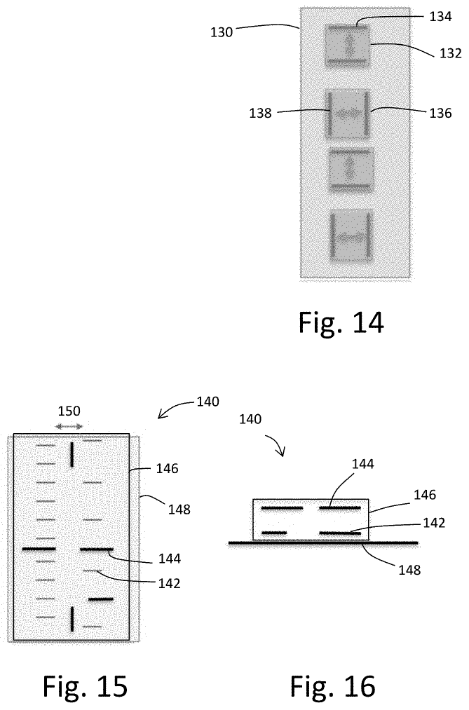

Another type of sensor that can be used on a pipe is shown in FIG. 14. FIG. 14 is a top view of a bi-directional sensor strip 130 with 4 sensors on. When installed on a pipe, 2 axial sensors 132 measure in an axial direction using contacts 134 and 2 circumferential sensors 136 measure in a circumferential direction using contacts 138. Connecting wires are not shown.

3D Sensors

Sensors can also be made 3-dimensional. FIGS. 15 and 16 show an example of a sensor 140 suitable for use on the leading edge of a wind turbine blade. A first set of contacts 142 in this embodiment is used to measure position of erosion along the blade. A second set of contacts 144 positioned in the Z direction away from the first set of contacts can be used to determine the position of erosion in the Z direction, for example by measuring resistance between a contact of the second set of contacts and contacts of the first set of contacts. Both sets of contacts are encased in a smart coating 146 on a film backing 148. The contacts can also be placed at varying Z positions rather than in sets at particular Z positions as shown here. The detection area in this embodiment comprises the volume of smart coating between contacts. In FIG. 15 an extent of the detection area 150 in the XY plane is shown. Measurement between top and bottom sensors through the coating film thickness gives a more accurate determination of the volume of coating lost and the location of the loss compared to a 2D sensor. The Z direction can be a thickness relative to a surface, or a third dimension of 3 dimensional volume of detection material.

SmartFoam

A sensor can be used to measure the effect of the force on a foam, for example in a seat cushion, so that a force can be applied in response to the detected pressure to make the foam confirm to a torso and minimize the pressure points exerted by the seat.

A SmartFoam is envisaged where the foam contains partially conductive materials such that the entire foam becomes a sensor, where either in a 2D or 3D configuration the foam is monitored by sensors for resistance changes. A partially conductive foam containing nano particles that gives a strong piezoresistive response to the stimulus is preferred.

The sensors could be wires embedded in the foam or distributed sensors that create a map of the foam. Sensing can be done using parallel sensors, or non-parallel sensors or a combination of the two in a 3D array. A configuration as shown in FIGS. 15 and 16 could be employed to monitor the foam.

These will give more precise locations of the defects. Thus, a map of the pressure exerted by the torso when seated is created, this data can be used to create a reactive seat that reduces pressure points by modifying the shape of the seat using robotic response mechanisms.

A concept design is a polyurethane foam pad featuring nanotechnology. The pad has sensors embedded in it to form a 3D sensing matrix, where change in resistance of the foam is based on the response to physical stimuli. This enables the detection of pressure over a 1 cm X/Y square and deflection of the foam pad in the Z direction as well. This pad will be installed in a wheelchair with robotic pressure pads set below it to respond to the pressure detected to form a more comfortable seat, eliminating pressure points that could form sores.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

1. A sensor for detecting an influence on a detection area of a surface, the sensor comprising:
a coating comprising an at least partially conductive material on the detection area of the surface;
an array of conductive contacts arranged outside of the detection area; and
a processor connected to the conductive contacts and configured to:
measure resistances between pairs of conductive contacts; and
produce an output indicating a location of the influence based on the pairs of resistances.

2. The sensor of claim 1 in which for each contact of the conductive contacts the processor is configured to measure resistances between multiple pairs of contacts that include that contact.

3. The sensor of claim 1 in which the coating has a thickness, and the conductive contacts include contacts arranged at different positions a thickness direction perpendicular to the surface, and the processor is configured to produce an output indicating a location of the influence in the thickness direction based on resistances measured between plural pairs of contacts, the contacts of the different pairs of the plural pairs not having the same combination of positions in the thickness direction.

4. The sensor of claim 1 further comprising plural switching circuitry elements each connected to one or more of the conductive contacts, the processor being connected to send signals to one or more of the plural switching circuitry elements to cause the one or more switching circuitry elements to close a circuit through a pair of conducting contacts and the partially conductive material on the detecting area to measure a resistance between the pair of conductive contacts.

5. A sensor for detecting an influence of a detection area of a sheet or volume of an at least partially conductive material, the sensor comprising:
plural conductive contacts arranged in electrical contact with the sheet or volume of the at least partially conductive material outside of the detection area;
a processor connected to the conductive contacts and configured to:
form or energize electrical circuits connecting pairs of the conductive contacts through the sheet or volume of at least partially conductive material;
measure an electrical property of the electrical circuits; and
produce an output indicating a location of the influence based on the measurements of the electrical property for the pairs of conductive contacts.

6. The sensor of claim 5 in which for each contact of the conductive contacts the processor is configured to form electrical circuits, of the electrical circuits, connecting multiple pairs of contacts that include that contact.

7. The sensor of claim 5 comprising plural nodes each with one or more associated conductive contacts of the plural conductive contacts, each node configured to at least partially form an electrical circuit of the electrical circuits by forming or energizing a connection to a conductive contact of the associated conductive contacts in response to a signal from the processor.

8. The sensor of claim 7 in which the signal is a wireless signal.

9. The sensor of claim 7 in which the nodes are configured to be powered by the signal.

10. The sensor of claim 5 in which the electrical property comprises a resistance of the at least partially conductive material.

11.-15. (canceled)

16. The sensor of claim 1 in which the at least partially conductive material comprises a nanocomposite material comprising one or more conductive nanoparticles embedded in a polymer.

17. (canceled)

18. The sensor of claim 16 in which the nanocomposite comprises either multiwall carbon nanotubes or graphene nanoplatelets or a combination of both of them embedded in a polymer.

19. The sensor of claim 1 in which the influence comprises at least one of A, B, C, D or E where:
A is a removal of a portion of the at least partially conductive material in the detection area;
B is a change in a crack in the at least partially conductive material in the detection area;
C is a change in temperature, the partially conductive material being thermally sensitive;
D is a strain on the coating, the at least partially conductive material being piezoresistive; and
E is an impingement of a fluid on the at least partially conductive material in the detection area, the at least partially conductive material being sensitive to the fluid.

20.-26. (canceled)

27. The sensor of claim 1 in which the surface is the surface of a chair, the at least partially conductive material is piezoresistive, and the influence is a strain caused by pressure of a person in the chair.

28. (canceled)

29. The sensor of claim 1 in which the detection area is at least a portion of a leading edge of a wind turbine blade, and the contacts are displaced from the leading edge.

30. The sensor of claim 1 in which the detection area is a portion of a surface of a blister pack.

31. The sensor of claim 30 in which the processor measures the resistance in response to input from a gravity or tilt sensor.

32. The sensor of claim 1 in which the processor is connected to the contacts via a wireless connection.

33.-34. (canceled)

35. A system for adjusting a chair to accommodate a person in the chair, the system comprising a sensor as claimed in claim 27 and actuators for adjusting the shape of the chair, and the processor being configured to produce a signal to cause the actuators to move, the signal being the output indicating a location of the influence or being based on the output indicating a location of the influence.

36. The sensor of claim 1 in which the output indicates locations of a start and an end of the influence in the detection area.
